(19) United States
(12) Patent Application Publication
(10) Pub. No.: US 2009/0297616 A1
(43) Pub. Date: Dec. 3, 2009

(54) Wound Care Composition

(76) Inventor: William Posten, Dallas, TX (US)

Correspondence Address: JACKSON WALKER LLP, 901 MAIN STREET, SUITE 6000, DALLAS, TX, 75202-3797, US

(21) Appl. No.: 12/472,090

(22) Filed: May 26, 2009

Related U.S. Application Data
(60) Provisional application No. 61/128,989, filed on May 28, 2008.

(51) Int. Cl. A61K 9/16; A61K 33/38; A61K 33/32

(52) U.S. Cl. 424/490; 424/618; 424/642

(57) ABSTRACT

The present invention relates to a wound care composition comprising petrolatum ointment and zinc oxide. The wound care composition is effective at accelerating the healing of a wound resulting from cutaneous surgery and for reducing the appearance of scars. The amount of zinc oxide in the wound care composition is preferably about 1% to about 10%, which produces effective wound healing properties as well as a cosmetically elegant composition having a pleasing feel on the skin.

### BACKGROUND

This application claims priority to U.S. Provisional Patent Application Ser. No. 61/128,989, entitled “Topical ointment to improve wound healing and scar appearance following surgery,” filed on May 28, 2008, the entire content of which is hereby incorporated by reference.

This invention pertains to a topical wound care composition for use in improving wound healing and scar appearance following cutaneous surgery.

Among the factors that may affect the outcomes of cutaneous surgery are the choices for post-surgical product use. Trials have examined the utility of tissue adhesives and post-treatment scar modification by laser and other means. Petrolatum ointment applied to wounds resulting from cutaneous surgery has also been shown to promote faster wound healing, compared to the wounds being exposed to air or covered by a bandage alone. It is presumed that these topical ointments improve the speed of wound healing by both trapping moisture in the wound, thus allowing wound healing matrix cells to flow more easily from one end of the incision to the other, and by acting as a barrier against microbial invasion to keep the wound clean and free of infections that can slow the healing process.

The presence of antibiotic compounds in topical ointments is not necessarily a primary factor in improving wound healing. It is presumed that many petrolatum ointments advertised for the promotion of wound healing exert their positive healing properties through the effect of the petrolatum vehicle more so than by the antibiotic compounds contained within. One problem with antibiotic ointments is that they tend to cause a large number of patients to display symptoms of allergic contact dermatitis due to the antibiotics, limiting their usefulness on wounds.

Use of an ointment to promote the healing of wounds resulting from cutaneous surgery, particularly on the face, is particularly problematic due to patients' desire for a cosmetically pleasing appearance and a suitable feel on the skin. The inclusion of additional ingredients in the ointment that might otherwise be helpful can also produce coloring, texture, and viscosity changes in the ointment that are not desirable to the patient.

Thus, it is desirable to have a wound care composition that not only effectively promotes wound healing and improves the appearance of scars after cutaneous surgery, but is also cosmetically elegant and has a pleasing feel on the skin.

### SUMMARY

The present invention pertains to a wound care composition that effectively improves the speed of wound healing and reduces the appearance of scars following cutaneous surgery. In particular, the wound care composition is made up of a petrolatum ointment and a particular percentage of zinc oxide.

The wound care composition has an optimal balance of petrolatum ointment and zinc oxide that makes it effective at improving wound healing and reducing scar formation while at the same time being cosmetically pleasing to patients. Because the composition includes the right balance of components in an optimized vehicle, it is unexpectedly and advantageously effective at improving wound healing by creating a barrier to microbial infection, thereby decreasing inflammation in the wound, to facilitate the speed of wound healing and decrease the incidence of scar formation.

### BRIEF DESCRIPTION OF DRAWINGS

FIG. 1 shows a chart comparing the scar scale measurements collected from patients receiving an experimental wound care composition formulation and patients receiving a standard post surgical therapy.

FIG. 2 shows a chart with the differences in average scar scale measurements between the patients receiving an experimental wound care composition formulation and patients receiving a standard post surgical therapy.

### DETAILED DESCRIPTION OF PREFERRED EMBODIMENTS

One aspect of the present invention is a wound care composition comprising petrolatum ointment and zinc oxide. The wound care composition is useful for accelerating the healing of wounds resulting from cutaneous surgery and for reducing the appearance of scars.

The wound care composition includes petrolatum ointment and zinc oxide. In preferred embodiments, the amount of zinc oxide present in the composition is about 1% to about 10% by weight. The amount of zinc oxide present in the composition is preferably about 10% by weight. Zinc oxide acts synergistically with the petrolatum ointment to provide anti-microbial and anti-fungal protection, as well as protection from sun exposure. The zinc oxide is also believed to provide some immunosuppressant characteristics.

Using a concentration of about 1% to about 10% of zinc oxide is critical for the wound care composition for several reasons. Higher concentrations produce undesirable properties, such as thickness and discoloration. To maintain the cosmetic elegance of the wound care composition, as well as its ease of application and removal and its pleasing feel on the skin, the concentration of zinc oxide should not exceed about 10%. In addition, as shown in the examples below, this amount of zinc oxide produces highly advantageous results when compared to the wound healing properties of petrolatum ointment alone.

The zinc oxide used in the wound care composition can be any kind of zinc oxide that is available from commercial sources. In preferred embodiments, the zinc oxide can be a coated or micronized form of zinc oxide. One preferred type of zinc oxide is Z-COTE® HP1 (BASF, New Jersey).

Micronized zinc oxide typically contains particles about 70 nm to 300 nm in size. The smaller the particle size, the easier it is to disperse the zinc oxide throughout the petrolatum ointment. In addition, smaller zinc oxide particles make the wound care composition appear more transparent and less white. This transparency is highly desirable to patients, particularly when the composition is being used on visible skin such as the face. Coated forms of zinc oxide are typically lightly coated with an inert barrier material such as triethoxycaprylylsilane, dimethicone, or silica. The addition of this coating makes the zinc oxide particles less photoreactive so they remain effective for a longer period of time. The petrolatum ointment used in the wound care composition can be any kind of petrolatum ointment that is available from commercial sources. In preferred embodiments, the petrolatum ointment can be white or red petrolatum. Preferred types of petrolatum ointment can be PENRECO® petrolatum products (Houston, Tex.). The petrolatum ointment acts as a moisture barrier to keep the wound moist.

In further embodiments, the wound care composition can contain additional optional ingredients. These optional ingredients can include anti-microbial and anti-fungal ingredients, preservatives, ingredients for the promotion of cell growth, additional moisture barriers, and additional ingredients for sun protection. More specifically, optional ingredients can include silver, hyaluronic acid, silicone, and titanium oxide, as well as other suitable ingredients known to those of skill in the art. The wound care composition can include any one or more of these additional optional ingredients, including combinations thereof. The appropriate concentrations of these optional ingredients need not be specified, as these types of ingredients are often included in topical ointments and suitable concentrations could be ascertained without undue effort depending on the effects desired.

The topical use of the wound care composition on cutaneous surgical wounds is highly effective at accelerating the healing of the wound and reducing the appearance of scars. This is of particular concern for facial wounds incurred during cutaneous surgeries, and also for those wounds incurred during Mohs surgery, which removes cancerous cells from the face and body. Facial wounds are problematic not only for the heightened concerns over scarring, but also for the concerns about the appearance and feel of any topical ointments applied to the face. Thus, the wound care composition described herein is ideally suited for treatment of such facial wounds, as well as all wounds incurred after cutaneous surgeries. Methods for accelerating the healing of wounds resulting from cutaneous surgeries and methods for reducing the appearance of scars resulting from cutaneous surgical wounds by applying the wound care composition to the cutaneous surgical wound are also aspects of the current invention.

Generally, the wound care composition is prepared by mixing the desired amount of zinc oxide with the desired amount of petrolatum. Any optional ingredients must be added to the initial mixture as well. This mixing must be done at a high enough rate to fully disperse the zinc oxide and produce a mostly transparent composition. The wound care composition can be sterilized either during production, after production, or by the addition of an anti-microbial ingredient or preservative.

In some instances, it is desirable to administer the wound care composition to the cutaneous surgical wound in combination with additional dermatologically acceptable carriers, which can be a solid, semi-solid, or a liquid. Useful solid carriers include, but are not limited to, finely divided solids such as talc, clay, microcrystalline cellulose, silica, alumina, and the like. Useful liquid carriers include, but are not limited to, water, alcohols, glycols, and water-alcohol/glycol blends in which the wound care composition could be dissolved or dispersed at effective levels, optionally with the aid of a surfactant. Adjuvants, such as fragrances and additional antimicrobial agents, can be added to optimize the properties for a given use. The resultant liquid compositions can be applied topically by absorbent pads, used to impregnate bandages and other dressings, or sprayed onto the affected area using pump-type or aerosol sprayers.

In some instances it is desirable to administer the wound care composition to the cutaneous surgical wound in a therapeutically effective amount for an amount of time that produces effective results. A “therapeutically effective amount” refers to that amount of the composition that results in achieving the desired effect. Thus, a therapeutically effective amount would be that amount of the composition that should be administered in order to achieve improved wound healing and reduction in the appearance of scars. Without limitations, it is presumed that the wound care composition should be applied in an amount sufficient to cover the cutaneous surgical wound in its entirety and should be re-applied regularly for a period of time that is about seven to about ten days after the wound is incurred, or at least long enough to observe substantial healing of the wound. The application of a bandage to cover the wound is necessary only to protect the wound from further injury.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### EXAMPLE 1

### Preparation

An example formulation of the wound care composition was prepared according to the following method. Zinc oxide (Z-COTE® HP1) and white petrolatum (PENRECO®) were mixed slowly at first, then at high shear to fully disperse the metal oxide and achieve homogenization. This step is necessary to achieve skin transparency. The mixing was performed at 54° C. for 10-15 minutes in order to get a uniform dispersion. This step was repeated three times, with the material that built up on the sides of the vessel being moved to the center with a spatula between each mixing cycle. A mixer/homogenizer with a mandrel attachment that produced approximately 5000 RMP was used to create enough shear stress to disperse the zinc oxide particles and produce a transparent product for use on the skin.

### EXAMPLE 2

### Comparison of Wound Care composition to Standard Post Surgical Therapy

A study was conducted to ascertain whether usage of an example formulation of the wound care composition can result in better functional and cosmetic outcomes than standard post surgical therapy. The study was a matched case control trial, comparing postsurgical outcomes following Mohs surgery for skin cancers on the face and body. Standard post surgical therapy included usage of topical petrolatum ointment (VASELINE®, Unilever, United Kingdom) for 4 weeks following surgery. Experimental therapy included substitution of topical petrolatum with a formulation of the wound care composition containing petrolatum ointment and 10% zinc oxide by weight.

In this study, the aesthetic and functional outcomes associated with 2 different types of post-surgical topical therapy were systematically examined. The primary means for reducing bias was using a case control method to control for the site of surgery and the type of closure performed. Bias was also reduced by having multiple surgeons participate in performing the surgeries, to mitigate the effect of surgeon specific factors. Each participating patient was seen at both a 1 week and 4 week time point to minimize the effect of patient-specific factors.

Informed consent was obtained from all subjects receiving post surgical cream. Historical patient controls had signed a consent form allowing use of pictures for research trials. Patients were recruited from a private practice dermatological surgery practice. Specifically, consecutive adult patients (ages 18-75 years) who were determined by a staff dermatologist to have Mohs surgery that required reconstructive repair were invited to participate. Exclusion criteria included prior patient experience of keloids or atypical scars, and known patient hypersensitivity to any of the ingredients of the creams used in the protocol. Consent was obtained from all willing participants before surgery.

In every case, techniques for repairing Mohs surgical defects consisted of standard elliptical excision repairs with a 1:2.5 width-length ratio along minimum skin tension lines. Undermining was performed in the mid-subcutis to a width of 1-2 cm circumferentially around the defect. Deep dermis and subcutis were closed with vertical deep sutures of 5.0 (face) and 4.0 (all other trunk and extremity sites) polyglactin 910 (VICRYL, Ethicon Inc, Somerville, N.J.) sutures placed 0.5 cm apart and no closer than 0.5 cm to each edge of the main axis (mean and median of 3 deep sutures per wound). Superficial closures were performed with 6.0 (face) and 5.0 (all other trunk and extremity)-caliber suture simple running polypropylene (PROLENE, Ethicon Inc) to be removed in 7 days.

Sample size determination: With a total of 20 patients in the study, the statistical power to detect a mean difference of 1 unit on the rating scale, assuming an SD of 0.50, was 80%. A2-tailed test and type I error rate of 5% were assumed. The results of this study confirmed the near accuracy ofthe standard deviation estimate and hence the adequacy of this sample size estimation. For the 1 week and 1 month time points, standard deviations of the study measures ranged from 0.33 to 0.74, with a median SD of 0.58. With the sample size and allocation described before, the statistical power to detect a mean difference in these measures of 0.54, assuming an SD of 0.58, was 80%. This effect size of 0.58 indicates that there is sufficient power to detect a mean difference of 1 unit on the rating scale, since the effect size of 0.58 represents an absolute difference in the means of the scales being measured.

The primary outcome measures were overall appearance of the scar measured on a 5-point ordinal scale (subscale of Hollander Scar Scale). Secondary outcome measures were 2 standardized questionnaire measures, the Vancouver Scar Scale and the Hollander Scar Scale. The Vancouver Scar Scale items measured vascularity, pliability, and height, each on a 3- to 6-point ordinal scale. Pigmentation was measured on a 3-point categorical scale.

Pigmentation
0=Normal color
1=Hypopigmentation
2=Hyperpigmentation

Vascularity
0=Normal
1=Pink (slight increase in blood supply)
2=Red (significant increase in blood supply)
3=Purple (excessive local blood supply)

Pliability
0=Normal
1=Supple (flexible with minimal resistance)
2=Yielding (giving way to pressure, offering moderate resistance, but does not behave as a solid scar mass)
3=Firm (solid/inflexible unit, not easily moved, resistant to manual pressure)
4=Banding (rope-like tissue that blanches with extension of scar, does not limit range of motion)
5=Contracture (permanent shortening of scar producing deformity or distortion; limits range of motion)

Height
0=Normal
1=_{—}2 mm
2=_{—}2 mm and _{—}5 mm
3=_{—}5 mm

The Hollander Scar Scale items measured presence and absence of step-off borders, contour irregularities, margin separation, edge inversion, and excessive distortion and overall appearance, each on a 5-point ordinal scale (0=absence, 1=trace, 2=mild, 3=moderate, and 4=severe). An additional questionnaire item was added to assess the degree of cream use.

Patient scars were assessed immediately after the surgery at 1 week and 1 month. At each time point, a blinded dermatologist was asked to rate each scar on the relevant outcome measures. Study participants and those administering the surgical interventions were not blinded to the therapy assignment because visual inspection of the surgical site revealed the assignment. Sham surgery to the blinded study participants was not considered because the surgical intervention was cutaneous and hence not amenable to concealment. Also, right after surgery and at each 1 week and 1 month time point, digital photographs with a single-lens reflex camera with spot flash (Nikon Coolpix; Nikon Co, Tokyo, Japan) were obtained under ambient fluorescent lights with standardized photography protocols.

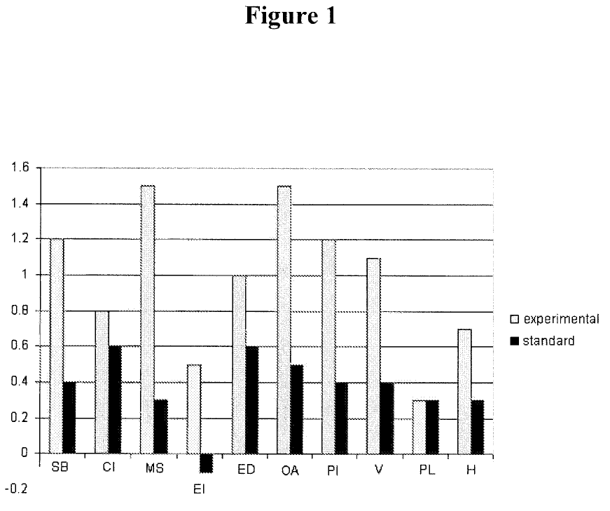

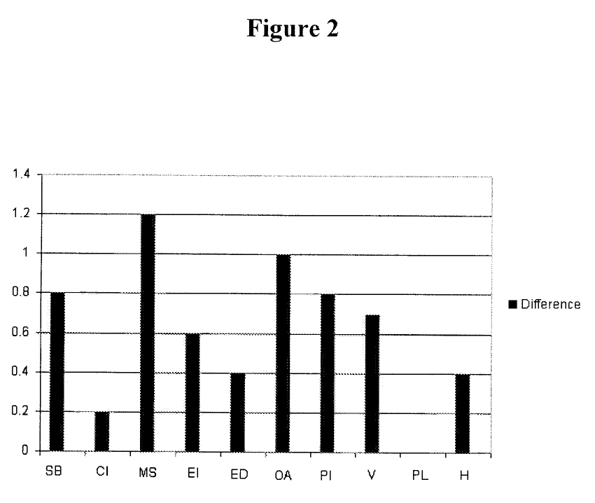

The live measurements by the blinded raters were used for data analysis. For reliability assessment, these live measurements were compared with post hoc ratings of the photographs at the end of the study. Blinded rater data were analyzed using linear model analysis. The ratings for the scales between 1 week and 4 week for the experimental wound care composition formulation are presented in Tables 1A and 1B below. Tables 2A and 2B below show the ratings for the scales between 1 week and 4 week for the standard therapy with case matched controls. FIG. 1 shows the average improvement from 1 week to 4 weeks in the scales comparing the experimental wound care composition formulation to the standard therapy. FIG. 2 shows the difference in the averages between the 2 therapies. In the figures, the measurements in the tables below are represented by the abbreviations appearing just below their names (e.g., Step off borders=“SB”).

TABLE 1A
Hollander Scar Scale

| | | Step off | Contour | Margin | Edge | Excessive | Overall |
|---|---|---|---|---|---|---|---|
| | | borders | irregularities | separation | inversion | distortion | appearance |
| | | (0-4) | (0-4) | (0-4) | (0-4) | (0-4) | (0-4) |
| Patient | Week | (“SB”) | (“CI”) | (“MS”) | (“EI”) | (“ED”) | (“OA”) |
| 1 | 1 | 2 | 3 | 1 | 1 | 2 | 3 |
| 1 | 4 | 1 | 1 | 0 | 0 | 0 | 1 |
| 2 | 1 | 1 | 0 | 2 | 1 | 1 | 3 |
| 2 | 4 | 0 | 0 | 0 | 1 | 0 | 1 |
| 3 | 1 | 2 | 1 | 2 | 1 | 1 | 3 |
| 3 | 4 | 1 | 1 | 0 | 1 | 1 | 2 |
| 4 | 1 | 2 | 2 | 1 | 1 | 1 | 2 |
| 4 | 4 | 1 | 1 | 0 | 1 | 1 | 1 |
| 5 | 1 | 2 | 3 | 4 | 4 | 3 | 3 |
| 5 | 4 | 1 | 1 | 1 | 2 | 2 | 1 |
| 6 | 1 | 2 | 1 | 1 | 0 | 2 | 2 |
| 6 | 4 | 1 | 1 | 0 | 0 | 1 | 1 |
| 7 | 1 | 2 | 2 | 1 | 1 | 2 | 2 |
| 7 | 4 | 1 | 1 | 0 | 1 | 1 | 1 |
| 8 | 1 | 2 | 2 | 2 | 1 | 1 | 1 |
| 8 | 4 | 1 | 2 | 0 | 0 | 0 | 0 |
| 9 | 1 | 2 | 1 | 1 | 1 | 1 | 3 |
| 9 | 4 | 0 | 1 | 0 | 1 | 0 | 1 |
| 10 | 1 | 2 | 2 | 1 | 1 | 2 | 2 |
| 10 | 4 | 0 | 0 | 0 | 0 | 0 | 0 |

TABLE 1B
Vancouver Scar Scale

| | | Pigmentation | Vascularity | Pliability | Height |
|---|---|---|---|---|---|
| | | (0-2) | (0-3) | (0-5) | (0-3) |
| Patient | Week | (“PI”) | (“V”) | (“PL”) | (“H”) |
| 1 | 1 | 2 | 2 | 3 | 1 |
| 1 | 4 | 1 | 1 | 2 | 0 |
| 2 | 1 | 2 | 1 | 3 | 1 |
| 2 | 4 | 0 | 0 | 3 | 0 |
| 3 | 1 | 2 | 2 | 2 | 1 |
| 3 | 4 | 2 | 1 | 2 | 0 |
| 4 | 1 | 2 | 2 | 2 | 0 |
| 4 | 4 | 1 | 1 | 2 | 0 |
| 5 | 1 | 2 | 2 | 1 | 2 |
| 5 | 4 | 0 | 1 | 1 | 1 |
| 6 | 1 | 2 | 2 | 2 | 1 |
| 6 | 4 | 1 | 1 | 2 | 1 |
| 7 | 1 | 2 | 1 | 2 | 1 |
| 7 | 4 | 0 | 1 | 2 | 1 |
| 8 | 1 | 1 | 2 | 2 | 1 |
| 8 | 4 | 0 | 0 | 2 | 1 |
| 9 | 1 | 2 | 2 | 2 | 2 |
| 9 | 4 | 0 | 0 | 2 | 0 |
| 10 | 1 | 0 | 1 | 2 | 1 |
| 10 | 4 | 0 | 0 | 0 | 0 |

TABLE 2A
Hollander Scar Scale

| | | Step off | Contour | Margin | Edge | Excessive | Overall |
|---|---|---|---|---|---|---|---|
| | | borders | irregularities | separation | inversion | distortion | appearance |
| | | (0-4) | (0-4) | (0-4) | (0-4) | (0-4) | (0-4) |
| Patient | Week | (“SB”) | (“CI”) | (“MS”) | (“EI”) | (“ED”) | (“OA”) |
| 11 | 1 | 2 | 2 | 0 | 1 | 1 | 1 |
| 11 | 4 | 2 | 1 | 0 | 1 | 1 | 1 |
| 12 | 1 | 1 | 1 | 0 | 0 | 1 | 0 |
| 12 | 4 | 1 | 1 | 0 | 0 | 1 | 0 |
| 13 | 1 | 1 | 1 | 0 | 0 | 0 | 1 |
| 13 | 4 | 1 | 1 | 0 | 0 | 0 | 1 |
| 14 | 1 | 2 | 2 | 2 | 0 | 2 | 2 |
| 14 | 4 | 1 | 1 | 0 | 1 | 0 | 0 |
| 15 | 1 | 2 | 1 | 1 | 2 | 1 | 2 |
| 15 | 4 | 2 | 1 | 1 | 1 | 1 | 2 |
| 16 | 1 | 1 | 1 | 1 | 0 | 0 | 1 |
| 16 | 4 | 1 | 1 | 1 | 0 | 0 | 1 |
| 17 | 1 | 2 | 2 | 2 | 1 | 2 | 2 |
| 17 | 4 | 2 | 2 | 1 | 1 | 2 | 2 |
| 18 | 1 | 2 | 3 | 1 | 1 | 2 | 2 |
| 18 | 4 | 0 | 1 | 1 | 1 | 0 | 1 |
| 19 | 1 | 2 | 2 | 1 | 0 | 2 | 2 |
| 19 | 4 | 1 | 1 | 1 | 1 | 1 | 1 |
| 20 | 1 | 2 | 2 | 1 | 1 | 2 | 2 |
| 20 | 4 | 2 | 1 | 1 | 1 | 1 | 1 |

TABLE 2B
Vancouver Scar Scale

| | | Pigmentation | Vascularity | Pliability | Height |
|---|---|---|---|---|---|
| | | (0-2) | (0-3) | (0-5) | (0-3) |
| Patient | Week | (“PI”) | (“V”) | (“PL”) | (“H”) |
| 11 | 1 | 0 | 1 | 2 | 1 |
| 11 | 4 | 0 | 1 | 2 | 1 |
| 12 | 1 | 0 | 1 | 2 | 1 |
| 12 | 4 | 0 | 0 | 2 | 1 |
| 13 | 1 | 0 | 1 | 2 | 1 |
| 13 | 4 | 0 | 1 | 2 | 1 |
| 14 | 1 | 1 | 2 | 3 | 1 |
| 14 | 4 | 1 | 1 | 2 | 0 |
| 15 | 1 | 2 | 2 | 3 | 1 |
| 15 | 4 | 1 | 2 | 2 | 1 |
| 16 | 1 | 0 | 1 | 2 | 1 |
| 16 | 4 | 0 | 0 | 2 | 1 |
| 17 | 1 | 2 | 1 | 2 | 1 |
| 17 | 4 | 2 | 1 | 2 | 1 |
| 18 | 1 | 2 | 1 | 2 | 2 |
| 18 | 4 | 1 | 1 | 2 | 1 |
| 19 | 1 | 1 | 1 | 2 | 2 |
| 19 | 4 | 1 | 1 | 1 | 1 |
| 20 | 1 | 2 | 2 | 2 | 2 |
| 20 | 4 | 0 | 1 | 2 | 2 |

The results of this study indicate that choice of post surgical therapy can affect short- to medium-term cosmesis and function, with the wound care composition formulation showing the most positive results. FIG. 2 demonstrates that the most significant differences can be seen in overall appearance, margin separation, step off borders, and pigmentation.

The results show that the wound care composition can improve scar appearance following cutaneous surgery when used for 4 weeks following surgery when compared to standard therapy. It appears to do this by several criteria on the standardized Hollander and Vancouver scar scales, notably, overall appearance, step off borders, margin separation and pigmentation. Limitations of this study include the short duration of the study of 4 weeks as it is known that scars continue to improve over the course of 2 years. However, it is the experience of the surgeons involved that appearance at 4 weeks can predict overall appearance at 2 years. The generalizability and external validity of this study is significant, given that the relevant surgical procedures were performed by 2 different surgeons, who had received clinical training at different programs.

What is claimed is:

1. A method for accelerating healing of wounds resulting from cutaneous surgery, comprising:
topically applying a wound care composition to the wounds resulting from cutaneous surgery, wherein the composition comprises petrolatum ointment and zinc oxide, and wherein the zinc oxide is present in a concentration of about 1% to about 10% by weight.

2. The method of claim 1, wherein the zinc oxide is present in a concentration of about 10% by weight.

3. The method of claim 1, wherein the zinc oxide is micronized.

4. The method of claim 1, wherein the zinc oxide is coated with an inert barrier material.

5. The method of claim 1, wherein the petrolatum ointment comprises white petrolatum, red petrolatum, or a combination thereof.

6. The method of claim 1, wherein the wound care composition further comprises one or more ingredients selected from the group consisting of antimicrobial ingredients, preservatives, ingredients for promotion of cell growth, moisture barriers, and combinations thereof.

7. The method of claim 1, wherein the wound care composition further comprises one or more ingredients selected from the group consisting of silver, hyaluronic acid, silicone, and combinations thereof.

8. The method of claim 1, wherein the wounds are facial wounds.

9. The method of claim 1, wherein the zinc oxide is evenly dispersed throughout the petrolatum ointment.

10. The method of claim 1, wherein the wound care composition is transparent.

11. The method of claim 1, wherein the wound care composition is applied in a therapeutically effective amount.

12. A wound care composition for accelerating healing of wounds resulting from cutaneous surgery and reducing appearance of scars resulting from the wounds, comprising:
petrolatum ointment; and
zinc oxide, wherein the zinc oxide is present in a concentration of about 1% to about 10% by weight.

13. The wound care composition of claim 12, wherein the zinc oxide is present in a concentration of about 10% by weight.

14. The wound care composition of claim 12, wherein the zinc oxide is micronized.

15. The wound care composition of claim 12, wherein the zinc oxide is coated with an inert barrier material.

16. The wound care composition of claim 12, wherein the petrolatum ointment comprises white petrolatum, red petrolatum, or a combination thereof.

17. The wound care composition of claim 12, wherein the wound care composition further comprises one or more ingredients selected from the group consisting of antimicrobial ingredients, preservatives, ingredients for promotion of cell growth, moisture barriers, and combinations thereof.

18. The wound care composition of claim 12, wherein the wound care composition further comprises one or more ingredients selected from the group consisting of silver, hyaluronic acid, silicone, and combinations thereof.

19. The wound care composition of claim 12, wherein the wounds are facial wounds.

20. The wound care composition of claim 12, wherein the zinc oxide is evenly dispersed throughout the petrolatum ointment.

21. The wound care composition of claim 12, wherein the wound care composition is transparent.

22. A method for reducing appearance of scars from wounds resulting from cutaneous surgery, comprising:
topically applying a wound care composition to the wounds resulting from cutaneous surgery, wherein the composition comprises petrolatum ointment and zinc oxide, and wherein the zinc oxide is present in a concentration of about 1% to about 10% by weight.

23. The method of claim 22, wherein the zinc oxide is present in a concentration of about 10% by weight.

24. The method of claim 22, wherein the zinc oxide is micronized.

25. The method of claim 22, wherein the zinc oxide is coated with an inert barrier material.

26. The method of claim 22, wherein the petrolatum ointment comprises white petrolatum, red petrolatum, or a combination thereof.

27. The method of claim 22, wherein the wound care composition further comprises one or more ingredients selected from the group consisting of antimicrobial ingredients, preservatives, ingredients for promotion of cell growth, moisture barriers, and combinations thereof.

28. The method of claim 22, wherein the wound care composition further comprises one or more ingredients selected from the group consisting of silver, hyaluronic acid, silicone, and combinations thereof.

29. The method of claim 22, wherein the wounds are facial wounds.

30. The method of claim 22, wherein the zinc oxide is evenly dispersed throughout the petrolatum ointment.

31. The method of claim 22, wherein the wound care composition is transparent.

32. The method of claim 22, wherein the wound care composition is applied in a therapeutically effective amount.
